(19) United States
(12) Patent Application Publication
(10) Pub. No.: US 2011/0201684 A1
(43) Pub. Date: Aug. 18, 2011

(54) EP2 Agonist from Non-Prostanoid Structures Designed as PGE2 Antagonists

(75) Inventors: David F. Woodward, Lake Forest, CA (US); Jenny W. Wang, Newport Coast, CA (US)

(73) Assignee: ALLERGAN, INC., Irvine, CA (US)

(21) Appl. No.: 13/095,372

(22) Filed: Apr. 27, 2011

Related U.S. Application Data
(63) Continuation of application No. 12/782,972, filed on May 19, 2010.
(60) Provisional application No. 61/179,971, filed on May 20, 2009.

(51) Int. Cl. A61K 31/195; C07C 229/34; A61P 27/02; A61P 27/06

(52) U.S. Cl. 514/567; 562/442

(57) ABSTRACT

A method of treating ocular hypertension, lowering intraocular pressure, pain or inflammation, comprising administering to a mammal a pharmaceutical composition of an EP₂-receptor agonist represented by

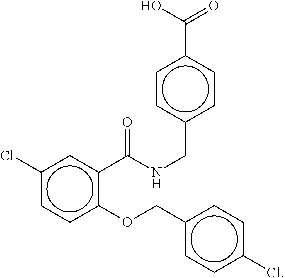

### RELATED APPLICATION

This application is a continuation of U.S. patent application Ser. No. 12/782,972, which claims the benefit of U.S. Provisional Application Ser. No. 61/179,971, filed May 20, 2009, the disclosure of which is hereby incorporated in its entirety herein by reference.

### FIELD OF THE INVENTION

The present invention relates to novel EP₂ receptor agonists that are useful for treating glaucoma, pain and inflammation and other conditions and indications in mammals.

### BACKGROUND OF THE INVENTION

Ocular hypotensive agents are useful in the treatment of a number of various ocular hypertensive conditions, such as post-surgical and post-laser trabeculectomy ocular hypertensive episodes, glaucoma, and as presurgical adjuncts.

Glaucoma is a disease of the eye characterized by increased intraocular pressure. On the basis of its etiology, glaucoma has been classified as primary or secondary. For example, primary glaucoma in adults (congenital glaucoma) may be either open-angle or acute or chronic angle-closure. Secondary glaucoma results from pre-existing ocular diseases such as uveitis, intraocular tumor or an enlarged cataract.

The underlying causes of primary glaucoma are not yet known. The increased intraocular tension is due to the obstruction of aqueous humor outflow. In chronic open-angle glaucoma, the anterior chamber and its anatomic structures appear normal, but drainage of the aqueous humor is impeded. In acute or chronic angle-closure glaucoma, the anterior chamber is shallow, the filtration angle is narrowed, and the iris may obstruct the trabecular meshwork at the entrance of the canal of Schlemm. Dilation of the pupil may push the root of the iris forward against the angle, and may produce pupillary block and thus precipitate an acute attack. Eyes with narrow anterior chamber angles are predisposed to acute angle-closure glaucoma attacks of various degrees of severity.

Secondary glaucoma is caused by any interference with the flow of aqueous humor from the posterior chamber into the anterior chamber and subsequently, into the canal of Schlemm. Inflammatory disease of the anterior segment may prevent aqueous escape by causing complete posterior synechia in iris bombe and may plug the drainage channel with exudates. Other common causes are intraocular tumors, enlarged cataracts, central retinal vein occlusion, trauma to the eye, operative procedures and intraocular hemorrhage.

Considering all types together, glaucoma occurs in about 2% of all persons over the age of 40 and may be asymptotic for years before progressing to rapid loss of vision. In cases where surgery is not indicated, topical β-adrenoreceptor antagonists have traditionally been the drugs of choice for treating glaucoma.

It has long been known that one of the sequelae of glaucoma is damage to the optic nerve head. This damage, referred to as “cupping”, results in depressions in areas of the nerve fiber of the optic disk. Loss of sight from this cupping is progressive and can lead to blindness if the condition is not treated effectively.

Unfortunately lowering intraocular pressure by administration of drugs or by surgery to facilitate outflow of the aqueous humor is not always effective in obviating damage to the nerves in glaucomatous conditions. This apparent contradiction is addressed by Cioffi and Van Buskirk [Surv. of Ophthalmol., 38, Suppl. p. S107-16, discussion S116-17, May 1994] in the article, “Microvasculature of the Anterior Optic Nerve”. The abstract states:
The traditional definition of glaucoma as a disorder of increased intraocular pressure (IOP) oversimplifies the clinical situation. Some glaucoma patients never have higher than normal IOP and others continue to develop optic nerve damage despite maximal lowering of IOP. Another possible factor in the etiology of glaucoma may be regulation of the regional microvasculature of the anterior optic nerve. One reason to believe that microvascular factors are important is that many microvascular diseases are associated with glaucomatous optic neuropathy.

Subsequent to Cioffi, et al., Matusi published a paper on the “Ophthalmologic aspects of Systemic Vasculitis” [Nippon Rinsho, 52 (8), p. 2158-63, August 1994] and added further support to the assertion that many microvascular diseases are associated with glaucomatous optic neuropathy. The summary states:
Ocular findings of systemic vasculitis, such as polyarteritis nodosa, giant cell angitis and aortitis syndrome were reviewed. Systemic lupus erythematosus is not categorized as systemic vasculitis, however its ocular findings are microangiopathic. Therefore, review of its ocular findings was included in this paper. The most common fundus finding in these diseases is ischemic optic neuropathy or retinal vascular occlusions. Therefore several points in diagnosis or pathogenesis of optic neuropathy and retinal and choroidal vasoocclusion were discussed. Choroidal ischemia was able to be diagnosed clinically, since fluorescein angiography was applied in these lesions. When choroidal arteries are occluded, overlying retinal pigment epithelium is damaged. This causes disruption of barrier function of the epithelium and allows fluid from choroidal vasculatures to pass into subsensory retinal spaces. This is a pathogenesis of serous detachment of the retina. The retinal arterial occlusion resulted in non-perfused retina. Such hypoxic retina released angiogenesis factors which stimulate retinal and iris neovascularizations and iris neovascularizations may cause neovascular glaucoma.

B. Schwartz, in “Circulatory Defects of the Optic Disk and Retina in Ocular Hypertension and High Pressure Open-Angle Glaucoma” [Surv. Ophthalmol., 38, Suppl. pp. S23-24, May 1994] discusses the measurement of progressive defects in the optic nerve and retina associated with the progression of glaucoma. He states:
Fluorescein defects are significantly correlated with visual field loss and retinal nerve fiber layer loss. The second circulatory defect is a decrease of flow of fluorescein in the retinal vessels, especially the retinal veins, so that the greater the age, diastolic blood pressure, ocular pressure and visual field loss, the less the flow. Both the optic disk and retinal circulation defects occur in untreated ocular hypertensive eyes. These observations indicate that circulatory defects in the optic disk and retina occur in ocular hypertension and open-angle glaucoma and increase with the progression of the disease.

Thus, it is evident that there is an unmet need for agents that have neuroprotective effects in the eye that can stop or retard the progressive damage that occurs to the nerves as a result of glaucoma or other ocular afflictions.

Prostaglandins were earlier regarded as potent ocular hypertensives; however, evidence accumulated in the last two decades shows that some prostaglandins are highly effective ocular hypotensive agents and are ideally suited for the long-term medical management of glaucoma. (See, for example, Starr, M. S. Exp. Eye Res. 1971, 11, pp. 170-177; Bito, L. Z. Biological Protection with Prostaglandins Cohen, M. M., ed., Boca Raton, Fla, CRC Press Inc., 1985, pp. 231-252; and Bito, L. Z., Applied Pharmacology in the Medical Treatment of Glaucomas Drance, S. M. and Neufeld, A. H. eds., New York, Grune & Stratton, 1984, pp. 477-505). Such prostaglandins include PGF_{2α}, PGF_{1α}, PGE₂, and certain lipid-soluble esters, such as C₁ to C₅ alkyl esters, e.g. 1-isopropyl ester, of such compounds.

### SUMMARY OF THE INVENTION

The present invention provides a method of treating ocular hypertension or lowering elevated intraocular pressure (IOP) or pain or inflammation, by administering to a mammal having ocular hypertension a therapeutically effective amount of a compound 4-{[5-chloro-2-(4-chloro-benzyloxy)-benzoylamino]-methyl}-benzoic acid represented by the formula:

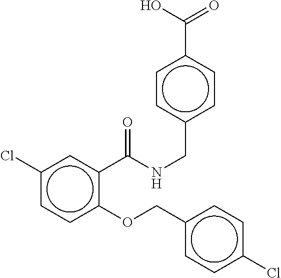

including any pharmaceutically-acceptable salts and prodrugs thereof.

In a further aspect, the present invention relates to an ophthalmic solution comprising a therapeutically effective amount of a compound of the above formula or a pharmaceutically-acceptable salt thereof, in admixture with a non-toxic, ophthalmically acceptable liquid vehicle, packaged in a container suitable for metered application.

In a still further aspect, the present invention relates to a pharmaceutical product, comprising
a container adapted to dispense its contents in a metered form; and
an ophthalmic solution or emulsion therein, as hereinabove defined.

### DETAILED DESCRIPTION OF THE INVENTION

The present invention relates to the use of a certain EP₂-receptor agonist.

The compound 4-{[5-chloro-2-(4-chloro-benzyloxy)-benzoylamino]-methyl}-benzoic acid used in accordance with the present invention are encompassed by the following structural formula:

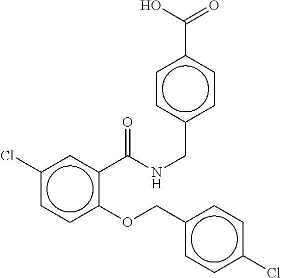

A pharmaceutically-acceptable salt is any salt which retains the activity of the parent compound and does not impart any deleterious or undesirable effect on the subject to whom it is administered and in the context in which it is administered. Of particular interest are salts formed with inorganic ions, such as sodium, potassium, calcium, magnesium and zinc.

Pharmaceutical compositions including the above compounds may be prepared by combining a therapeutically effective amount of at least one compound according to the present invention, or a pharmaceutically-acceptable salt thereof, as an active ingredient, with conventional ophthalmically acceptable pharmaceutical excipients, and by preparation of unit dosage forms suitable for topical ocular use. The therapeutically efficient amount typically is between about 0.0001 and about 5% (w/v), preferably about 0.001 to about 1.0% (w/v) in liquid formulations.

For ophthalmic application, preferably solutions are prepared using a physiological saline solution as a major vehicle. The pH of such ophthalmic solutions should preferably be maintained between 4.5 and 8.0 with an appropriate buffer system, a neutral pH being preferred but not essential. The formulations may also contain conventional, pharmaceutically-acceptable preservatives, stabilizers and surfactants.

Preferred preservatives that may be used in the pharmaceutical compositions of the present invention include, but are not limited to, benzalkonium chloride, chlorobutanol, thimerosal, phenylmercuric acetate and phenylmercuric nitrate. A preferred surfactant is, for example, Tween 80. Likewise, various preferred vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose cyclodextrin and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar vein, an ophthalmically acceptable antioxidant for use in the present invention includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. The preferred chelating agent is edentate disodium, although other chelating agents may also be used in place of or in conjunction with it.

The ingredients are usually used in the following amounts:

| Ingredient | Amount (% w/v) |
|---|---|
| active ingredient | about 0.001-5 |
| preservative | 0-0.10 |
| vehicle | 0-40 |
| tonicity adjuster | 0-10 |
| buffer | 0.01-10 |
| pH adjuster | q.s. pH 4.5-8.0 |
| antioxidant | as needed |
| surfactant | as needed |
| purified water | as needed to make |
| | 100% |

The actual dose of the active compounds of the present invention depends on the specific compound, and on the condition to be treated; the selection of the appropriate dose is well within the knowledge of the skilled artisan.

The ophthalmic formulations for use in the method of the present invention are conveniently packaged in forms suitable for metered application, such as in containers equipped with a dropper, to facilitate application to the eye. Containers suitable for dropwise application are usually made of suitable inert, non-toxic plastic material, and generally contain between about 0.5 and about 15 ml solution. One package may contain one or more unit doses.

Especially preservative-free solutions are often formulated in non-resealable containers containing up to about ten, preferably up to about five units doses, where a typical unit dose is from one to about 8 drops, preferably one to about 3 drops. The volume of one drop usually is about 20-35 μl.

The invention is further illustrated by the following examples which are illustrative of a specific mode of practicing the invention and are not intended as limiting the scope of the claims.

### Example I

Measurement of intraocular pressure studies in dogs will involve applanation pneumatonometry performed in Beagle dogs of both sexes. The animals will remain conscious throughout the study and will be gently restrained by hand. Compound 4-{[5-chloro-2-(4-chloro-benzyloxy)-benzoylamino]-methyl}-benzoic acid will be administered topically to one eye using a dropper bottle to deliver approximately a 35 μl volume, the other eye received vehicle (1% polysorbate 80 in 5 mM Tris HCl) as a control. Proparacaine at 0.25% was used for corneal anesthesia during tonometry. Intraocular pressure will be determined just before drug administration and at 2, 4, 6 hours thereafter on each day of the 5 day study. Measurement of ocular surface hyperemia will be performed immediately before each of the intraocular pressure readings. Ocular surface hyperemia grading will be semi-quantitative and assessed according to a 5 point scoring scale used for clinical evaluations: 0=none; 0.5=trace; 1=mild; 2=moderate; and 3=severe. It is expected that administering compound 4-{[5-chloro-2-(4-chloro-benzyloxy)-benzoylamino]-methyl}-benzoic acid will significantly reduce intraocular pressure in the eye.

1) A pharmaceutical composition wherein the composition comprises an EP₂-receptor agonist compound of the following structure:

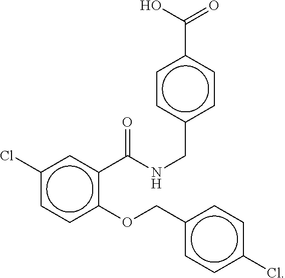

2. A method of treating one of the following conditions selected from the group consisting of ocular hypertension, lowering intraocular pressure, pain and inflammation, the method comprising administering to a mammal in need of such treatment a pharmaceutical composition comprising a compound represented by:

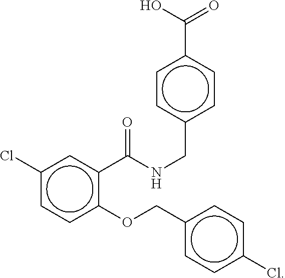

3. The method of claim 2, wherein the composition comprises a pharmaceutically acceptable salt of compound

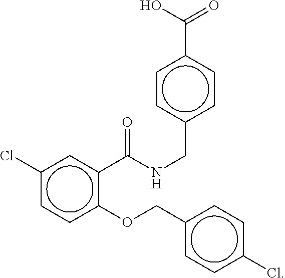

4. The method of claim 2, wherein the composition comprises a pharmaceutically acceptable ophthalmic emulsion of compound [chemical structure]
